(19) United States
(12) Patent Application Publication
(10) Pub. No.: US 2012/0232248 A1
(43) Pub. Date: Sep. 13, 2012

(54) THERAPEUTIC AGENT FOR PULMONARY SMALL CELL CARCINOMA

(75) Inventors: Shigeru Kotake, Tokyo (JP); Yuki Nanke, Tokyo (JP); Manabu Kawamoto, Tokyo (JP); Toru Yago, Tokyo (JP); Hisashi Yamanaka, Tokyo (JP)

(73) Assignee: TOKYO WOMEN'S MEDICAL UNIVERSITY, Tokyo (JP)

(21) Appl. No.: 13/395,009

(22) PCT Filed: Jan. 25, 2010
(86) PCT No.: PCT/JP2010/050894

§ 371 Date: Mar. 8, 2012

(30) Foreign Application Priority Data
Sep. 10, 2009 (JP) 2009-209355

(51) Int. Cl. C07K 14/435; C07K 7/06; C07K 5/08; C07K 7/08

(52) U.S. Cl. 530/324; 530/326; 530/327; 530/329; 530/331

(57) ABSTRACT

The invention aims to clarify further functions of a TCTA protein-derived peptide and provide a novel application of the peptide. The invention provides a therapeutic agent for pulmonary small cell carcinoma or a cell growth inhibitor of pulmonary small cell carcinoma, comprising any peptide described in SEQ ID NOs 1 to 5.

### TECHNICAL FIELD

The present invention relates to a therapeutic agent for pulmonary small cell carcinoma and a cell growth inhibitor of pulmonary small cell carcinoma, comprising a particular peptide.

### BACKGROUND ART

The inventors of the invention have previously made experiments to identify novel factors in the rheumatoid synovial membrane that promote or inhibit the differentiation of human osteoclasts, and then found that a novel peptide derived from T-cell leukemia translocation-associated gene (TCTA) protein can work to inhibit the differentiation of human osteoclasts (JP 2008-148566 A and Kotake S, et al., Bone. 2009; 45: 627-639).

Although partial defect of the TCTA gene in the pulmonary small cell carcinoma is reported (Aplan PD et al., Cancer Res. 1995; 55: 1917-1921), other functions of the TCTA protein than the above-mentioned function found by the inventors still have not been clarified.

### SUMMARY OF INVENTION

### Technical Problem

An object of the invention is to provide a novel use of a peptide derived from the TCTA protein by clarifying another function of the peptide.

### Solution to Problem

As a result of extensive studies, the inventors of the present invention found that the 29-mer peptide of the TCTA protein can inhibit proliferation of the human pulmonary small cell carcinoma. The invention has been accomplished based on the above-mentioned finding.

Namely, the invention provides a therapeutic agent for pulmonary small cell carcinoma, comprising a peptide selected from the group consisting of the following (I) to (VI):

(I)
Gly Gln Asn,
(II)
Gly Gln Asn Gly Ser Thr,
(III)
Gly Gln Asn Gly Ser Thr Pro Asp Gly Ser Thr His Phe Pro Ser Trp Glu Met Ala
Ala Asn Glu Pro Leu Lys Thr His Arg Glu,
(IV)
Gly Gln Asn Gly Ser Thr Pro Asp Gly Ser Thr His Phe Pro Ser Trp Glu Met Ala
Ala Asn,
(V)
Gly Gln Asn Gly Ser Thr Pro Asp Gly Ser Thr His Phe,
and
(VI)
Pro Gly Leu Gly Gly Gln Asn Gly Ser Thr Pro Asp Gly Ser Thr His Phe.

The invention also provides a use of the above-mentioned peptide for manufacturing a therapeutic agent for pulmonary small cell carcinoma.

Further, the invention provides a cell growth inhibitor of pulmonary small cell carcinoma, comprising a peptide selected from the group consisting of the following (I) to (VI):

(I)
Gly Gln Asn,
(II)
Gly Gln Asn Gly Ser Thr,
(III)
Gly Gln Asn Gly Ser Thr Pro Asp Gly Ser Thr His Phe Pro Ser Trp Glu Met Ala
Ala Asn Glu Pro Leu Lys Thr His Arg Glu,
(IV)
Gly Gln Asn Gly Ser Thr Pro Asp Gly Ser Thr His Phe Pro Ser Trp Glu Met Ala
Ala Asn,
(V)
Gly Gln Asn Gly Ser Thr Pro Asp Gly Ser Thr His Phe,
and
(VI)
Pro Gly Leu Gly Gly Gln Asn Gly Ser Thr Pro Asp Gly Ser Thr His Phe.

The invention also provides a use of the above-mentioned peptide for manufacturing a cell growth inhibitor of pulmonary small cell carcinoma.

### Effects of Invention

The above-mentioned peptide is capable of inhibiting the proliferation of cells of human pulmonary small cell carcinoma, so that the invention can provide an excellent therapeutic agent for pulmonary small cell carcinoma and an excellent cell growth inhibitor of pulmonary small cell carcinoma. In particular, the above-mentioned agents are useful as a therapeutic agent for metastatic pulmonary small cell carcinoma because the agent has an inhibitory effect on proliferation of the cancer cells and an inhibitory effect on bone resorption by the osteoclasts in the treatment of bone metastasis from lung cancer. In more particular, the agents of the invention may be used as biologics when the agents comprise the peptide from TCTA protein.

### BRIEF DESCRIPTION OF DRAWINGS

FIG. 1 is a graph showing the inhibitory action of peptide A on proliferation of the cells of human pulmonary small cell carcinoma.

FIG. 2 is a graph showing the inhibitory action of a scrambled peptide on proliferation of the cells of human pulmonary small cell carcinoma.

FIG. 3 is a graph showing the inhibitory action on proliferation of the cells of human pulmonary small cell carcinoma, where the concentrations of the peptide A and the scrambled peptide are respectively 0 μg/mL, and 10 μg/mL or 10 μg/mL, and 0 μg/mL.

### DESCRIPTION OF EMBODIMENTS

The peptide used in the invention has any one of the following sequences:

(I)
Gly Gln Asn,
(II)
(SEQ ID No. 1)
Gly Gln Asn Gly Ser Thr,
(III)
(SEQ ID No. 2)
Gly Gln Asn Gly Ser Thr Pro Asp Gly Ser Thr His Phe Pro Ser Trp Glu Met Ala
Ala Asn Glu Pro Leu Lys Thr His Arg Glu,
(IV)
(SEQ ID No. 3)
Gly Gln Asn Gly Ser Thr Pro Asp Gly Ser Thr His Phe Pro Ser Trp Glu Met Ala
Ala Asn,
(V)
(SEQ ID No. 4)
Gly Gln Asn Gly Ser Thr Pro Asp Gly Ser Thr His Phe,
and
(VI)
(SEQ ID No. 5)
Pro Gly Leu Gly Gly Gln Asn Gly Ser Thr Pro Asp Gly Ser Thr His Phe
Gly Gln Asn.

Hereinafter the peptides of Sequence ID Nos. 2, 3, 4 and 5 will also be referred to as peptides A, A2, B and C, respectively. Of those peptides, the peptides A, A2 and B are preferable, and the peptides A and A2 are more preferable, and the peptide A is most preferable. The base sequences coding the peptides having the Sequence ID Nos. 1 to 5 are respectively shown as Sequence ID Nos. 6 to 10.

The peptide used in the invention can be obtained by isolation and purification from the synovial membrane tissue of patients with rheumatoid arthritis, using the known methods such as gel filtration, ion-exchange chromatography, reversed phase chromatography, mass spectrum and the like. The peptide used in the invention can also be synthesized in accordance with the general chemical synthetic methods of amino acids, for example, the Fmoc method. Alternatively, the synthesis of the peptide can be achieved by using a commercially available apparatus for synthesizing amino acids.

Preferably, the peptide used in the invention may be derived from the TCTA protein. When the peptide used in the invention is a peptide from TCTA protein, the agents of the invention can be formulated as biologics. A lot of biologics have been used in the clinical field not only as anticancer agents, but also antibody preparations, receptor preparations, hormone preparations and the like. However, any biologics related to the TCTA protein have not been known, so it is expected to use novel biologics based on the invention.

The pulmonary small cell carcinoma, i.e., the target disease to be treated by the invention, accounts for 20% of all cases of lung cancer. The pulmonary small cell carcinoma, which is largely linked to smoking, often develops from the bronchus on the side of the nerve center. The pulmonary small cell carcinoma is frequently diagnosed as already advanced when discovered. This is because the degree of malignancy is high, the cancer grows rapidly, and the cancer tends to induce early metastasis, lymphogenous metastasis or hematogenous metastasis, to other organs such as bone or brain. The present invention is particularly useful as a therapeutic agent for metastatic pulmonary small cell carcinoma because the agent has an inhibitory effect on proliferation of the cancer cells and an inhibitory effect on bone resorption by the osteoclasts in the treatment of bone metastasis from lung cancer.

The above-mentioned peptide is administered as it is or as a form of various pharmaceutical compositions. The dosage forms of such pharmaceutical compositions may include, for example, tablets, powders, pills, granules, capsules, suppositories, solutions, sugar-coated pills, depot preparations, syrups and the like. Those dosage forms can be produced by the conventional methods using generally used adjuvants for formulation.

For example, the tablets can be obtained by mixing the above-mentioned peptide as the active ingredient of the invention together with known auxiliary additives including an inactive diluent such as lactose, calcium carbonate, calcium phosphate or the like; a binder such as gum arabic, corn starch, gelatin or the like; a swelling agent such as alginic acid, corn starch, pregelatinized starch or the like; a sweetener such as sucrose, lactose, saccharin or the like; a flavoring agent such as peppermint, Gaultheria adenothrix (Akamono) oil, cherry or the like; a wetting agent such as magnesium stearate, talc, carboxymethyl cellulose or the like; an excipient for soft gelatin capsules and suppositories, such as fats, waxes, semi-solid and liquid polyols, natural oils, hardened oils or the like; an excipient for solutions, such as water, alcohols, glycerols, polyols, sucrose, inverted sugar, glucose, vegetable oils or the like.

When the therapeutic agent or inhibitor according to the invention may be formulated into tablets, for example, 100 mg of any one of the peptides with the Sequence ID Nos. 1 to 5 may be mixed with 900 mg of lactose.

The dose of the therapeutic agent or inhibitor according to the invention is determined depending on the administration route, the period of treatment, the patient's age and weight, and other factors. For example, by local administration or systemic administration using bronchoscope, the adult oral dose may generally be 300 mg to 1 g per day; and the adult parenteral dose may generally be 300 μg to 300 mg per day.

### Example 1

### Inhibition of Proliferation of Cancer Cells

### 1. Peptide

As the peptide, the 29-mer peptide A (Sequence ID No. 2) derived from the TCTA protein, which contained extracellular domain GQN, was used.

The scrambled peptide (with Sequence ID No. 11: Ser Pro Phe Thr Gly Thr Lys Gly Ser Trp Asn Glu Thr Ala His Pro Asp His Gly Asn Glu Glu Arg Gln Ala Pro Met Ser Leu) corresponding to the peptide A was used as a control.

The synthesis of the above-mentioned peptide A and the scrambled peptide corresponding to the peptide A was committed to Toray Research Center (located in Kamakura City).

### 2. Materials and Methods

The human pulmonary small cell carcinoma cell line RERF-LC-MA, the human prostate cancer cell line PC-3, and the human breast cancer cell line MCF-7 were used as the cancer cells.

A 96-well plate was inoculated with the above-mentioned cells at a density of 0.6×10³ to 1.0×10³ cells per well, and then cultured at 37° C. for three days. Then, cell proliferation was quantified using the Cell Proliferation Kit (XTT based).

Next, the above-mentioned peptides were separately added to the culture system at concentrations of 0 μg/mL, 1 μg/mL, 5 μg/mL and 10 μg/m, followed by incubation at 37° C. for three days. After completion of the incubation, the number of cancer cells was counted.

### 3. Results

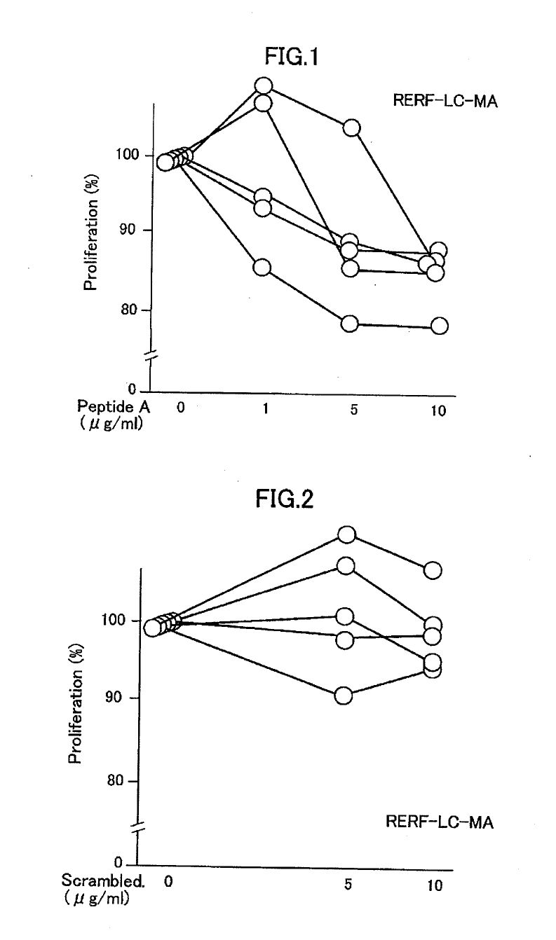

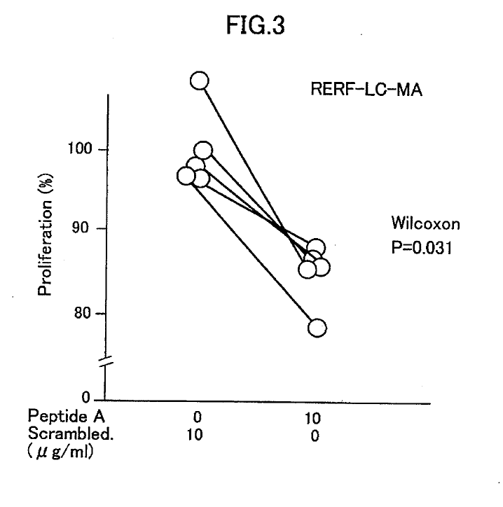

When the peptide A was added at a concentration of 5 μg/mL or 10 μg/m, the proliferation of the RERF-LC-MA cells was reduced to about 85%, which was statistically significant (p=0.031) as compared with the case of the scrambled peptide as the control, as shown in FIGS. 1 to 3. However, there was almost no inhibitory effect on proliferation of the PC-3 cells and the MCF-7 cells (not shown).

### Example 2

The peptides were added to the cancer cells and osteoclasts to determine a change in the expression of each molecular group resulting from the interaction between the peptide and the cancer cells or between the peptide and osteoclasts by confirming the expression of mRNA using quantitative polymerase chain reaction (qPCR).

### 1. Peptides

The peptide A and the scrambled peptide as described in Example 1 were used.

2. Molecule Groups and Cells where the Molecule Groups are Expressed

The following molecule groups were used which are expressed in the cancer cells and/or osteoclasts as reported in Yoko Morioka et al.: “The roles of MMPs and RECK in cancer progression”, Cell Technology. Vol. 28, No. 7, 659-679 (2009).

TABLE 1

| Expressing Molecule Groups | Cells |
|---|---|
| VEGFRI (Flt-1) | Osteoclasts |
| RECK | Cancer cells and osteoclasts |
| MMP9 | Cancer cells and osteoclasts |
| MMP14 (MT1-MMP) | Cancer cells and osteoclasts |

As the cancer cells, those as shown in Example 1 were used.

### 3. Method

The cancer cells and the osteoclasts were separately inoculated into a 6-well plate at a density of 1×10⁵ to 2×10⁵ cells per well. Twenty-four hours later, the medium was changed to D-MEM (1% FCS, 2 mM L-glu). Another 24 hours later, the medium was changed, and at the same time the peptide A or the control was added. After 24 hours, the cells were collected and extraction of the RNA was conducted.

### 4. Results

There was no significant difference between the peptide A and the scrambled peptide in the expression of mRNA of the VEGFR1, RECK, MMP9 and MMP14 in the osteoclasts. The above-mentioned results show that the expression levels of the above-mentioned molecule groups have no relation to the effect of peptide A on the cancer cells and osteoclasts.

With respect to the cancer cells, on the other hand, the addition of peptide A reduced the GAPDH mRNA expression only in the RERF-LC-MA cell. The above-mentioned results show that the peptide A works to reduce the proliferation of RERF-LC-MA cells by inhibiting the expression of GAPDH gene as a so-called house-keeping gene.

As can be seen from the examples shown above, the above-mentioned peptide inhibited the proliferation of human pulmonary small cell carcinoma cell line. The differentiation of human osteoclast can be inhibited by the peptide at low concentrations. The above-mentioned peptides derived from human tissue are expected to be used for treatment of the metastatic pulmonary small cell carcinoma because those peptides have an inhibitory effect on proliferation of the cancer cells and also an inhibitory effect on bone resorption by the osteoclasts in the treatment of bone metastasis from lung cancer.

1. A therapeutic agent for pulmonary small cell carcinoma, comprising a peptide selected from the group consisting of the following (I) to (VI):
(I)
Gly Gln Asn,
(II)
Gly Gln Asn Gly Ser Thr,
(III)
Gly Gln Asn Gly Ser Thr Pro Asp Gly Ser Thr His Phe Pro Ser Trp Glu Met Ala Ala
Asn Glu Pro Leu Lys Thr His Arg Glu,
(IV)
Gly Gln Asn Gly Ser Thr Pro Asp Gly Ser Thr His Phe Pro Ser Trp Glu Met Ala Ala
Asn,
(V)
Gly Gln Asn Gly Ser Thr Pro Asp Gly Ser Thr His Phe,
and
(VI)
Pro Gly Leu Gly Gly Gln Asn Gly Ser Thr Pro Asp Gly Ser Thr His Phe.

2. The therapeutic agent for pulmonary small cell carcinoma of claim 1, wherein the peptide is represented by Sequence ID No. 2.

3. The therapeutic agent for pulmonary small cell carcinoma of claim 1, wherein the peptide is derived from T-cell leukemia translocation-associated gene (TCTA) protein.

4. The therapeutic agent for pulmonary small cell carcinoma of claim 1, wherein the pulmonary small cell carcinoma is metastatic pulmonary small cell carcinoma.

5. Use of the peptide of claim 1 for manufacturing a therapeutic agent for pulmonary small cell carcinoma.

6. A cell growth inhibitor for pulmonary small cell carcinoma, comprising a peptide selected from the group consisting of the following (I) to (VI):
(I)
Gly Gln Asn,
(II)
Gly Gln Asn Gly Ser Thr,
(III)
Gly Gln Asn Gly Ser Thr Pro Asp Gly Ser Thr His Phe Pro Ser Trp Glu Met Ala Ala
Asn Glu Pro Leu Lys Thr His Arg Glu,
(IV)
Gly Gln Asn Gly Ser Thr Pro Asp Gly Ser Thr His Phe Pro Ser Trp Glu Met Ala Ala
Asn,
(V)
Gly Gln Asn Gly Ser Thr Pro Asp Gly Ser Thr His Phe,
and
(VI)
Pro Gly Leu Gly Gly Gln Asn Gly Ser Thr Pro Asp Gly Ser Thr His Phe.

7. The cell growth inhibitor for pulmonary small cell carcinoma of claim 6, wherein the peptide is represented by Sequence ID No. 2.

8. The cell growth inhibitor for pulmonary small cell carcinoma of claim 6, wherein the peptide is derived from T-cell leukemia translocation-associated gene (TCTA) protein.

9. Use of the peptide of claim 6 for manufacturing a cell growth inhibitor for pulmonary small cell carcinoma.


SEQUENCE LISTING (11 sequences)

SEQ ID NO: 1 - protein, 6 aa, Homo sapiens
GQNGST

SEQ ID NO: 2 - protein, 29 aa, Homo sapiens
GQNGSTPDGSTHFPSWEMAANEPLKTHRE

SEQ ID NO: 3 - protein, 21 aa, Homo sapiens
GQNGSTPDGSTHFPSWEMAAN

SEQ ID NO: 4 - protein, 13 aa, Homo sapiens
GQNGSTPDGSTHF

SEQ ID NO: 5 - protein, 17 aa, Homo sapiens
PGLGGQNGSTPDGSTHF

SEQ ID NO: 6 - DNA, 18 bp, Homo sapiens
ggtcagaatggatccacg

SEQ ID NO: 7 - DNA, 87 bp, Homo sapiens
ggtcagaatggatccacgcctgatggctccacgcatttcccttcgtgggaaatggcagcaaacgaacctctcaaaacccacagagaa

SEQ ID NO: 8 - DNA, 63 bp, Homo sapiens
ggtcagaatggatccacgcctgatggctccacgcatttcccttcgtgggaaatggcagcaaac

SEQ ID NO: 9 - DNA, 39 bp, Homo sapiens
ggtcagaatggatccacgcctgatggctccacgcatttc

SEQ ID NO: 10 - DNA, 51 bp, Homo sapiens
ccaggtctgggtggtcagaatggatccacgcctgatggctccacgcatttc

SEQ ID NO: 11 - protein, 29 aa, Homo sapiens
SPFTGTKGSWNETAHPDHGNEERQAPMSL